(19) United States
(12) Patent Application Publication
(10) Pub. No.: US 2019/0169116 A1
(43) Pub. Date: Jun. 6, 2019

(54) PROCESS FOR PREPARATION OF CARMUSTINE

(71) Applicant: INTAS PHARMACEUTICALS LTD., Gujarat, Ahmedabad (IN)

(72) Inventors: Gobind Singh KAPKOTI, Ahmedabad (IN); Nirmal KUMAR, Ahmedabad (IN); Mettilda LOURDUSAMY, Quebec (CA); Ioan-Iosif RADU, Quebec (CA); Aakash Maheshkumar SHAH, Ahmedabad (IN); Hitesh Manubhai MAKWANA, Ahmedabad (IN); Mohamed Zuber Abdulhaq SHAIKH, Ahmedabad (IN); Yogesh Naranbhai VAGHASIYA, Ahmedabad (IN)

(21) Appl. No.: 16/304,057

(22) PCT Filed: Mar. 24, 2017
(86) PCT No.: PCT/IB2017/051706

§ 371 Date: Nov. 21, 2018

(30) Foreign Application Priority Data
Apr. 16, 2016 (IN) 201621013299

(51) Int. Cl. C07C 275/68; C07C 273/18

(52) U.S. Cl. CPC C07C 275/68; C07C 273/1854

(57) ABSTRACT

The present invention relates to an improved process for preparation of carmustine (I). The present invention also relates to preparation of 1,3-bis(2-chloroethyl)urea (II) an intermediate used in preparation of carmustine.

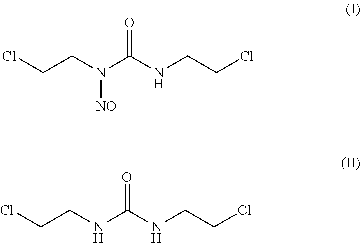

### RELATED APPLICATIONS

This application is related to Indian Provisional Application No. IN 201621013299 filed 16 Apr. 2016 and is incorporated herein in its entirety.

### FIELD OF THE INVENTION

The present invention relates to an improved process for preparation of carmustine (I).

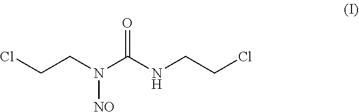

The present invention also relates to preparation of 1,3-bis(2-chloroethyl)urea (II)

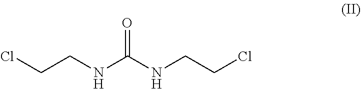

an intermediate used in preparation of carmustine.

### BACKGROUND OF THE INVENTION

“Carmustine” [154-93-8] is a medication used mainly for chemotherapy and sometimes for immunosuppression before transplant. It is a nitrogen mustard β-chloro-nitrosourea compound used as an alkylating agent. As a dialkylating agent, BCNU is able to form interstrand crosslinks in DNA, which prevents DNA replication and DNA transcription. Carmustine having an empirical formula of C₅H₉Cl₂N₃O₂ and a molecular weight of 214.06 g/mol. Carmustine is the international commonly accepted name for 1,3-bis-(2-chloroethyl)-1-nitrosourea and has the structure of formula (I)

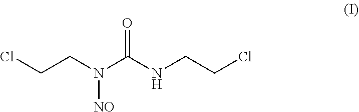

BiCNU® (carmustine for injection) is one of the nitrosoureas used in the treatment of certain neoplastic diseases.

Process for preparation of carmustine and its urea intermediate of formula (II) is known from various prior arts for example U.S. Pat. Nos. 4,028,410, 6,096,923, FR2589860, R056999A2 and U.S. Pat. No. 4,128,639.

The reported processes to prepare carmustine can be summarized as following scheme:

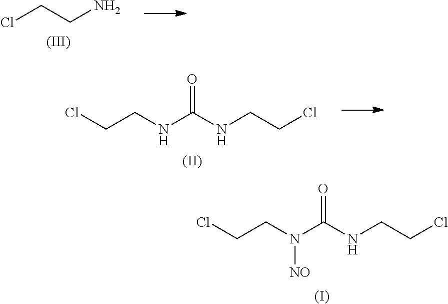

All the reported processes use phosgene for preparation of intermediate of formula (II).

Phosgene is highly toxic substance that exists as a gas at room temperature. According to the National Institute for Occupations Safety and Health (NIOSH), a toxic level of phosgene that can place a person's life and well-being in jeopardy can be as low as 2 parts per million.

In view of the hazardous nature of phosgene, it is highly desirable to replace phosgene in the process for preparation of carmustine.

The present invention provides a process to prepare carmustine wherein the use of hazardous reagents specifically phosgene is completely avoided. Hence the process of present invention is safe, simple and industrial friendly.

### OBJECTS OF THE INVENTION

The main object of the present invention is to provide a process for preparation of carmustine of formula (I)

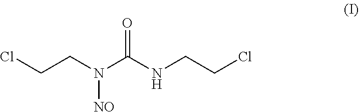

Another object of the present invention is to provide a process for preparation of carmustine (I), wherein the process includes preparing 1,3-bis(2-hydroxyethyl)urea (II) an intermediate in preparation of carmustine in absence of phosgene.

Yet another object of the invention is to provide a process for preparation of carmustine wherein 1,3-bis[2-hydroxyethyl]urea is prepared from 2-chloroethan-1-amine or 2-aminoethan-1-ol in presence of 1,1-carbonyldiimidazole, triphosgene or phenyl chloroformate.

Yet another object of present invention is to provide a process for preparation of carmustine comprising a step of converting 2-chloroethylamine to 1,3-bis[2-hydroxyethyl]urea (II) in absence of phosgene.

Yet another object of present invention is to provide a process for preparation of carmustine comprising a step of reacting 2-chloroethylamine with 1,1′-carbonyldiimidazole to obtain 1,3-bis[2-hydroxyethyl]urea (II).

Yet another object of present invention is to provide a process for preparation of carmustine comprising steps of:
a) reacting 2-chloroethylamine with 1,1′-carbonyldiimidazole, triphosgene or phenyl chloroformate;
b) converting 1,3-bis(2-hydroxyethyl)urea (II) to carmustine (I).

Yet another object of present invention is to provide a process for preparation of carmustine comprising steps of:
a) reacting 2-chloroethylamine with 1,1′-carbonyldiimidazole, triphosgene or phenyl chloroformate;
b) subjecting 1,3-bis(2-hydroxyethyl)urea (II) obtained in step a); to nitrosation; and
c) isolating carmustine (I).

Yet another object of present invention is to provide a process for preparation of carmustine comprising steps of:
a) reacting 2-chloroethylamine with 1,1′-carbonyldiimidazole;
b) reacting 1,3-bis(2-hydroxyethyl)urea (II) obtained in step a) with sulphuric acid and sodium nitrite; and
c) isolating carmustine (I).

### SUMMARY OF THE INVENTION

The main aspect of the present invention is to provides a process for preparation of carmustine (I)

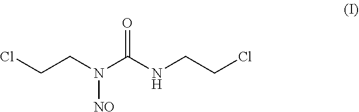

In an aspect, the present invention provides a process to prepare carmustine (I) wherein the process comprises a step of converting 2-chloroethyl amine (III) or its salt to 1,3-bis(2-chloroethyl)urea (II) in absence of phosgene.

In an aspect, the present invention provides a process to prepare carmustine (I) wherein the process comprises a step of reacting 2-chloroethyl amine (III) or its salt with 1,1′-carbonyldiimidazole to obtain 1,3-bis(2-chloroethyl)urea (II).

In an aspect, the present invention provides a process to prepare carmustine (I) wherein the process comprises steps of:
a) converting 2-chloroethyl amine (III) or its salt to 1,3-bis(2-chloroethyl)urea (II) in absence of phosgene;
b) converting 1,3-bis(2-chloroethyl) urea to carmustine.

In another aspect, the present invention provides a process to prepare carmustine wherein the process comprises:
a) reacting 2-chloroethyl amine or its salt with a reagent selected from 1,1′-carbonyldiimidazole or triphosgene or phenyl chloroformate to obtain 1,3-bis (2-chloroethyl)urea (II);
b) reacting 1,3-bis(2-chloroethyl)urea with nitrosating reagent to obtain carmustine.

In another aspect, the present invention provides a process to prepare carmustine wherein the process comprises steps of:
a) reacting 2-chloroethyl amine or its salt with a reagent selected from 1,1′-carbonyldiimidazole or triphosgene or phenyl chloroformate to obtain 1,3-bis(2-chloroethyl)urea (II);
b) reacting 1,3-bis(2-chloroethyl)urea in presence of sulphuric acid and sodium nitrite; and
c) isolating carmustine (I).

### DETAILED DESCRIPTION OF THE INVENTION

The main embodiment of the present invention is to provide a process for preparation of carmustine (I).

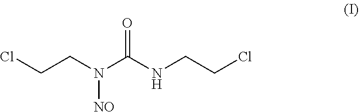

In another embodiment, the present invention provides a process wherein 2-chloroethylamine or its salts are converted to 1,3-bis(2-chloroethyl)urea (II) in absence of phosgene.

Processes known in the art generally uses phosgene for above conversion. Phosgene is very hazardous chemical and need to be avoided on large scale preparation. Present invention provides a process for preparation of carmustine wherein the step of preparing intermediate (II) entirely avoids use of phosgene, and thus make the overall process simple yet efficient and environment friendly.

In another embodiment the present invention provides a process to prepare carmustine as depicted by following scheme:

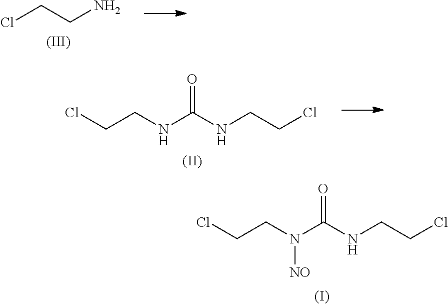

Conversion of 2-chloroethyl amine of formula (III) to an intermediate of formula (II) can be carried out in presence of a reagent selected from 1,1′-carbonyldiimidazole, triphosgene or phenyl chloroformate.

This reaction can be carried out in presence of base and solvent. Base for the purpose of above conversion can be selected from any suitable organic or inorganic base. Preferably the reaction to prepare urea intermediate is carried out in presence of triethylamine.

Solvent can be selected from any organic solvent or mixture of solvents suitable for the reaction. This reaction is generally carried out at temperature in the range from 5°−50° C.

In next step of the process intermediate of formula (II) is subjected to nitrosation.

Carmustine is nitroso-urea compound so the synthesis involves first step of obtaining urea intermediate i.e. intermediate of formula (II) and then converting urea intermediate to carmustine by nitrosation.

The nitrosation reaction can be carried out in presence of sulphuric acid and sodium nitrite. The reaction is carried out in presence of solvent. The reaction can be carried out in presence of single solvent, mixture of solvents or in two phase solvent system. Preferably the nitrosation reaction is carried out in presence of dichloromethane and water. Nitrosation is generally carried out at a temperature in range of −5° C. to 10° C.

The nitrosation reaction can be carried out by using suitable nitrosating agent selected from dinitrogen trioxide, sodium nitrite and aq. HCl, sodium nitrite and H₂SO₄, sodium nitrite and HCOOH and sodium nitrite and CH₃COOH.

The final product i.e. carmustine can be isolated by removal of solvent from reaction mixture or separating the layers and isolating the product from organic layer.

In an embodiment the product can be isolated by removing the solvent from the reaction mixture, followed by addition of suitable solvent to the residue to isolate the product.

In another embodiment carmustine can be isolated by removing solvent from organic layer, adding suitable solvent to the residue to dissolve carmustine, followed by addition of anti-solvent to isolate carmustine. Solvents used to dissolve residue (i.e. carmustine) can be selected form group consisting of ether such as diethyl ether, methyl tert-butyl ether, alkyl acetate such as ethyl acetate, aromatic hydrocarbon such as toluene and alkanol such as ethanol, isopropanol. Anti-solvent used to isolate carmustine can be selected from group consisting of alkanes such as heptane, hexane, or pentane, cycloalkanes such as cyclohexane and water.

In general the solvents used for the purpose of present invention i.e. used for conducting the reaction or isolating the product can be selected form any suitable solvent such as alkanol, ketone, ether, hydrocarbon, chlorinated solvents, water, aprotic solvents, alkanes or mixture thereof.

The process of the present invention uses non-hazardous and easily available reagents for preparation of carmustine, thus, makes the process commercially viable.

Following examples are provided by way of illustration only and should not be construed as limiting the scope of the invention in any manner.

### EXAMPLES

### Example 1: Preparation of 1,3-(bis(2-chloroethyl)urea (II)

2-chloroethyl amine hydrochloride (20 g) and triethylamine (90 ml) were added to a round bottom flask. The resultant reaction mixture was cooled to 10-20° C., to this 15 g of 1,1′-carbonyldiimidazole (CDI) was added, the reaction mixture was stirred for 2 hrs at 35-40° C. After completion of reaction the reaction mixture was cooled to 20-25° C., solvent was distilled of to obtain residue. 100 ml water was added to the residue and the mixture was filtered, the solid thus obtained was dried to give 13 g of title compound.

### Example 2: Preparation of Carmustine (I)

Water (80 ml) was taken in a round bottom flask, cooled to 0-5° C., to this sulphuric acid (38 g) was added. This was followed by addition of 80 ml of methylene dichloride and 10 g of 1,3-bis(2-chloroethyl)urea (II). The reaction mixture was stirred while maintaining the temperature at about 0-5° C., to this sodium nitrite solution (24 g sodium nitrite in 120 ml water) was added. The reaction mixture was stirred at the same temperature for 2 hrs, after completion of reaction, layers separated, organic layer was washed with sodium sulfate, and the solvent was distilled off. To the residue a mixture of n-heptane and methyl tertiary butyl ether was added. Reaction mixture was stirred for 1 hr, filtered and the solid thus obtained was dried to give 12 g of title compound.

1. A process for preparation of carmustine (I) 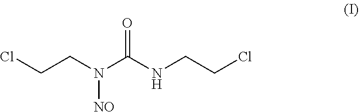
comprising a step of:
a) converting 2-chloroethylamine (III) to 1,3-bis(2-chloroethyl)urea (II) 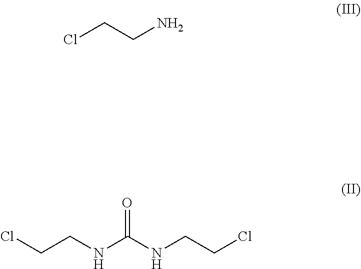
in absence of phosgene.

2. A process for preparation of carmustine (I) 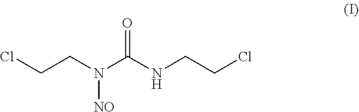
comprising steps of:
a) converting 2-chloroethylamine (III) to 1,3-bis(2-chloroethyl)urea (II) 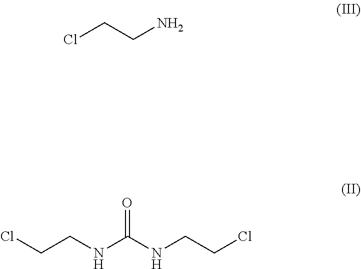
in absence of phosgene.
b) converting urea of formula (II) to carmustine (I).

3. The process according to claim 1, wherein step (a) is carried out in presence of a reagent selected from group consisting of 1,1-carbonyldiimidazole, triphosgene and phenyl chloroformate.

4. The process according to claim 2, wherein step (b) is carried out in presence of nirosating reagent.

5. The process according to claim 4, wherein nitrosating reagent is selected from group consisting of di-nitrogen trioxide, sodium nitrite and acid.

6. The process according to claim 5, wherein acid is selected from hydrochloric acid, sulphuric acid, formic acid and acetic acid.

7. The process according to claim 4, wherein nitrosating reagent is sodium nitrite and sulphuric acid.

8. A process for preparation of carmustine (I) comprising steps of:
a) reacting 2-chloroethyl amine (III) or its salt with a reagent selected from 1,1′-carbonyldiimidazole, triphosgene and phenyl chloroformate in presence of base to obtain 1,3-bis (2-chloroethyl) urea (II);
b) reacting 1,3-bis(2-chloroethyl)urea with nitrosating reagent in presence of solvent to obtain carmustine;
wherein step (a) is carried out in absence of phosgene.

9. The process according to claim 8, wherein base used in step (a) is triethylamine.

10. The process according to claim 8, wherein solvent used in step (b) is dichloromethane and water.

11. The process according to claim 2, wherein step (a) is carried out in presence of a reagent selected from group consisting of 1,1-carbonyldiimidazole, triphosgene and phenyl chloroformate.
